(19) United States
(12) Patent Application Publication
(10) Pub. No.: US 2006/0036109 A1
(43) Pub. Date: Feb. 16, 2006

(54) Tropylideneamines and use thereof

(76) Inventors: Hansjorg Grutzmacher, Dielsdorf (CH); Torsten Buttner, Eisenberg (DE); Pascal Maire, Liestal (CH); Maaike Ramseier, Zurich (CH); David Scheschkewitz, Wurzburg (DE); Theo Zweifel, Zug (CH)

Correspondence Address: LANXESS CORPORATION, 111 RIDC PARK WEST DRIVE, PITTSBURGH, PA, 15275-1112, US

(21) Appl. No.: 11/146,372

(22) Filed: Jun. 6, 2005

(30) Foreign Application Priority Data
Jun. 8, 2004 (DE) 1020040277710

(51) Int. Cl. C07F 15/00; C07C 211/42

(52) U.S. Cl. 556/137; 564/427

(57) ABSTRACT

The present invention relates to tropylideneamines, to a process for their preparation and to the use thereof in catalysis.

### FIELD OF THE INVENTION

The present invention relates to tropylideneamines, to a process for their preparation and to the use thereof in catalysis.

### BACKGROUND OF THE INVENTION

Deblon (Thesis No. 13920, ETH Zurich, 2000, Chapter 5) and Maire (Thesis No. 14396, ETH Zurich, 2001) disclose that transition metal complexes of olefin-phosphine compounds are particularly suitable for homogeneous catalytic reactions, especially hydrogenations and hydrosilylations.

However, it would be advantageous for industrial applications to be able to dispense with the often expensive and oxidation-sensitive phosphines. There is therefore a need to provide a catalyst system and ligands suitable therefor, which does not need the use of phosphines and shows good performance in catalytic reactions.

### SUMMARY OF THE INVENTION

Compounds of the formula (I) have now been found 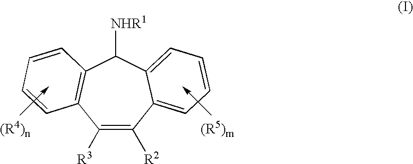
wherein
R¹ is either a radical of the formula (II) H 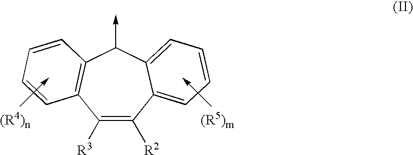
   or is a radical of the formula (III) 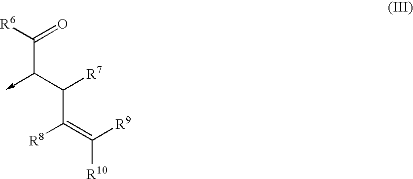
      where, in the formulae (I), (II) and (III),
the arrows each indicate the bond of the overall radical to the nitrogen atom or, when they point into the middle of an aromatic system, a bond of the particular radicals to the aromatic skeleton in any position,
R² and R³ are each independently hydrogen, cyano, fluorine, chlorine, bromine, iodine, C₁-C₁₈-alkyl, C₄-C₂₄-aryl, C5-C₂₅-arylalkyl, CO₂M where M may be an alkali metal ion or an optionally organic ammonium ion, CONH₂, SO₂N(R¹¹)₂ where R¹¹ is hydrogen, C₁-C₁₂-alkyl, C₄-C₁₄-aryl or C₅-C₁₅-arylalkyl, SO₃M or are radicals of the formula (IV),
   T-Het-R¹² (IV)
   wherein
   T is absent or is carbonyl,
   Het is oxygen or NR^{11,}
   R¹² is C₁-C₁₈-alkyl, C₄-C₂₄-aryl or C₅-C₂₅-arylalkyl or N(R¹²)₂ as a whole is a 5- or 6-membered cyclic amino radical and
n and m are each independently 0, 1, 2 or 3 and
R⁴ and R⁵ are each independently selected from the group of fluorine, chlorine, bromine, iodine, nitro, free or protected formyl, C₁-C₁₂-alkyl, C₁-C₁₂-alkoxy, C₁-C₁₂-haloalkoxy, C₁-C₁₂-haloalkyl, C₄-C₁₄-aryl, C₅-C₁₅-arylalkyl or radicals of the formula (V)
   L-Q-T-W (V)
   wherein, each independently,
   L is absent or is C₁-C₈-alkylene or C₂-C₈-alkenylene and
   Q is absent or is oxygen, sulfur or NR¹¹,
   T is a carbonyl group and
   W is R¹¹, OR¹¹, NHR¹² or N(R¹²)₂, where N(R¹²)₂ as a whole may also be a 5- or 6-membered cyclic amino radical,
      or radicals of the formulae (VIa-g)
      L-W (VIa) L-SO₂-W (VIb)
      L-NR¹²SO₂R¹² (VIc) L-SO₃Z (VId)
      L-PO₃Z₂ (VIe) L-COZ (VIf)
      L-CN (VIg)
      wherein L, Q, W and R¹² are each as defined under formula (IV) and Z is hydrogen or M
and, in formula (III),
R⁶ is OR¹¹ or N(R¹¹)₂, where N(R¹¹)₂ together may also be a 5- or 6-membered cyclic amino radical,
R⁷ and R⁸ are each independently hydrogen, C₁-C₈-alkyl, C₄-C₁₀-aryl, C₅-C₁₁-arylalkyl or C₂-C₈-alkenyl and
R⁹ and R¹⁰ are each independently hydrogen, C₁-C₈-alkyl, C₄-C₁₄-aryl, C₅-C₁₁-arylalkyl or C₂-C₈-alkenyl or
R⁷ and R9 or R⁸ and R¹⁰, in each case together, are C₃-C₁₂-alkylene or C₃-C₁₂-alkenylene.

### DETAILED DESCRIPTION OF THE INVENTION

Depending on the substitution, the compounds of the formula (I) may also be chiral. The invention also encompasses any stereoisomers which occur and any mixtures thereof. In the context of the invention, the terms stereoisomerically enriched (enantiomerically enriched or diastereomerically enriched) mean stereoisomerically pure (enantiomerically pure or diastereomerically pure) compounds or mixtures of stereoisomers (enantiomers or diastereomers) wherein one stereoisomer (enantiomer or diastereomer) is present in a larger proportion than another or the other. Stereoisomerically enriched means, for example and with preference, a content of one stereoisomer or 50% to 100% by weight, more preferably 70% to 100% by weight and most preferably 90 to 100% by weight, based on the sum of the particular stereoisomers.

The scope of the invention encompasses all combinations of radical definitions, parameters and illustrations above and listed below, in general or within areas of preference, with one another, i.e. also any combinations between the particular areas and areas of preference.

In the context of the invention, unless specifically stated otherwise, aryl is carbocyclic aromatic radicals, preferably phenyl, naphthyl, phenanthrenyl and anthracenyl, or heteroaromatic radicals wherein no, one, two or three skeleton carbon atoms per cycle, but at least one skeleton carbon atom in the entire molecule, is/are substituted by heteroatoms which are selected from the group of nitrogen, sulfur and oxygen, preferably pyridinyl, oxazolyl, thiophenyl, benzofuranyl, benzothiophenyl, dibenzofuranyl, dibenzothiophenyl, furanyl, indolyl, pyridazinyl, pyrazinyl, imidazolyl, pyrimidinyl and quinolinyl.

In addition, the carbocyclic, aromatic radicals or heteroaromatic radicals may be substituted by up to five identical or different substituents per cycle. For example and with preference, the substituents are selected from the group of bromine, fluorine, chlorine, nitro, cyano, free or protected formyl, free or protected hydroxyl, C₁-C₁₂-alkyl, C₁-C₁₂-haloalkyl, C₁-C₁₂-alkoxy, C₁-C₁₂-haloalkoxy, C₄-C₁₄-aryl, for example phenyl, C₅-C₁₅-arylalkyl, for example benzyl, di(C₁-C₁₂-alkyl)amino, (C₁-C₁₂-alkyl)amino, CO(C₁-C₁₂-alkyl), OCO(C₁-C₁₂-alkyl), NHCO(C₁-C₁₂-alkyl), N(C₁-C₈-alkyl)CO(C₁-C₁₂-alkyl), CO(C₄-C₁₄-aryl), OCO(C₄-C₁₄-aryl), NHCO(C₄-C₁₄-aryl), N(C₁-C₈-alkyl)CO(C₄-C₁₄-aryl), COO-(C₁-C₁₂-alkyl), COO-(C₄-C₁₄-aryl), CON(C₁-C₁₂-alkyl)₂ or CONH(C₁-C₁₂-alkyl)CO₂M, CONH₂, SO₂NH₂, SO₂N(C₁-C₁₂-alkyl)₂, SO₃M where M is in each case optionally substituted ammonium, lithium, sodium or potassium.

For example and with preference, aryl is phenyl or naphthyl which may be further substituted by no, one, two or three radicals per cycle which is/are selected from the group of fluorine, chlorine, cyano, C₁-C₈-alkyl, C₁-C₈-perfluoroalkyl, C₁-C₈-alkoxy, phenyl, benzyl, di(C₁-C₁₂-alkyl)amino, CO(C₁-C₁₂-alkyl), COO-(C₁-C₁₂-alkyl), CON(C₁-C₁₂-alkyl)₂ or SO₂N(C₁-C₁₂-alkyl)₂.

More preferably, aryl is phenyl which may be further substituted by no, one or two radicals per cycle which are selected from the group of fluorine, chlorine, cyano, C₁-C₄-alkyl, C₁-C₄-perfluoroatkyl, C₁-C₄-alkoxy, phenyl or SO₂N(C₁-C₄-alkyl)₂.

In the context of the invention, the definition and the areas of preference also apply analogously to aryloxy substituents and the aryl moiety of an arylalkyl radical.

In the context of the invention, unless specifically stated otherwise, protected formyl is a formyl radical which is protected by conversion to an aminal, acetal or a mixed aminal acetal, and the aminals, acetals and mixed aminal acetals may be acyclic or cyclic.

For example and with preference, protected formyl is a 1,1-(2,4-dioxycyclopentanediyl) radical.

In the context of the invention, unless specifically stated otherwise, protected hydroxyl is a hydroxyl radical which is protected by conversion to a ketal, acetal or a mixed aminal acetal, and the acetals and mixed aminal acetals may be acyclic or cyclic.

For example and with preference, protected hydroxyl is a tetrahydropyranyl radical (O-THP).

In the context of the invention, unless specifically stated otherwise, alkyl, alkylene, alkoxy, alkenyl and alkenylene are a straight-chain, cyclic, branched or unbranched alkyl, alkylene, alkoxy, alkenyl and alkenylene radical respectively, each of which may optionally be further substituted by C₁-C₄-alkoxy in such a way that each carbon atom of the alkyl, alkylene, alkoxy, alkenyl or alkenylene radical bears at most one heteroatom selected from the group of oxygen, nitrogen and sulfur.

The same applies to the alkylene moiety of an arylalkyl radical.

For example, in the context of the invention, C₁-C₄-alkyl is preferably methyl, ethyl, 2-ethoxyethyl, n-propyl, isopropyl, n-butyl, tert-butyl, C₁-C₈-alkyl is additionally, for example, n-pentyl, cyclohexyl, n-hexyl, n-heptyl, n-octyl or isooctyl, C₁-C₁₂-alkyl is further additionally, for example, norbornyl, adamantyl, n-decyl and n-dodecyl and C₁-C₁₈-alkyl is still further additionally n-hexadecyl and n-octadecyl.

For example, in the context of the invention, C₁-C₈-alkylene is preferably methylene, 1,1-ethylene, 1,2-ethylene, 1,1-propylene, 1,2-propylene, 1,3-propylene, 1,1-butylene, 1,2-butylene, 2,3-butylene and 1,4-butylene, 1,5-pentylene, 1,6-hexylene, 1,1-cyclohexylene, 1,4-cyclohexylene, 1,2-cyclohexylene and 1,8-octylene.

For example, in the context of the invention, C₁-C₄-alkoxy is preferably methoxy, ethoxy, isopropoxy, n-propoxy, n-butoxy and tert-butoxy, and C₁-C₈-alkoxy is additionally cyclohexyloxy.

For example, in the context of the invention, C₂-C₈-alkenyl is preferably ally, 3-propenyl and 4-butenyl.

For example, in the context of the invention, C₃-C₈-alkenylene is preferably 2-butenediyl.

In the context of the invention, unless specifically stated otherwise, haloalkyl and haloalkoxy are a straight-chain, cyclic, branched or unbranched alkyl and alkoxy radical respectively, each of which is substituted singly, multiply or fully by halogen atoms. Radicals which are fully substituted by fluorine are referred to as perfluoroalkyl and perfluoroalkoxy respectively.

For example, in the context of the invention, C₁-C₁₂-haloalkyl is trifluoromethyl, 2,2,2-trifluoroethyl, chloromethyl, fluoromethyl, bromomethyl, 2-bromoethyl, 2-chloroethyl, nonafluorobutyl, n-perfluorooctyl or n-perfluorododecyl.

The preferred substitution patterns for compounds of the formula (I) are defined below:
R¹ is preferably either a radical of the formula (II),
   wherein R² and R³ are each independently hydrogen, fluorine, iodine, C₁-C₁₂-alkyl, C₁-C₁₂-alkoxy, C₄-C₁₀-aryl or radicals of the formula (7V) wherein T is in turn carbonyl and Het is oxygen or NR¹¹, and wherein
   n and m are each independently 0 or 1 and
   R⁴ and R⁵ are each independently selected from the group of fluorine, bromine, nitro, C₁-C₄-alkyl, C₁-C₄-alkoxy or radicals of the formulae (VIb) and (VIg)
   or a radical of the formula (11),
   wherein R⁶ is OR¹¹ or N(R¹¹)₂, where N(R¹¹)₂ together may also be a 5- or 6-membered cyclic amino radical, R⁷ and R⁸ are each independently hydrogen, C₁-C₄-alkyl, or C₂-C₈-alkenyl, and R⁹ and Rlo are each independently hydrogen, C₁-C₄-alkyl, C₄-C₁₄-aryl or C₂-C₈-alkenyl, or R⁸ and R¹⁰ are alternatively in each case together C₃-C8-alkenediyl.
R¹ is either more preferably a radical of the formula (II)
   wherein R² and R³ are each independently hydrogen, C₁-C₁₂-alkyl, C₁-C₁₂-alkoxy, C₄-C₁₀-aryl
   n and m are each identically 0 or 1 and
   R⁴ and R⁵ are each identically selected from the group of fluorine and radicals of the formulae (VIb) and (VIg)
   or a radical of the formula (III)
   wherein R⁶ is OR¹¹, R¹ and R⁹ are each independently hydrogen or C₁-C₄-alkyl, and R⁸ and R¹⁰ in each case together are C₃-C₈-alkenediyl.
R¹ is most preferably either a radical of the formula (II)
   wherein R² and R³ are each independently hydrogen, C₁-C₁₂-alkyl, C₁-C₁₂-alkoxy or C₄-C₁₀-aryl and
   n and m are each 0
   or a radical of the formula (III)
   wherein R⁶ is OR¹¹, R⁷ and R⁹ are each identically hydrogen, and R⁸ and R¹⁰ are in each case together C₃-C₈-alkenediyl.
R² and R³ are preferably each independently hydrogen, fluorine, iodine, C₁-C₁₂-alkyl, C₄-C₁₀-aryl or radicals of the formula (IV) wherein T is in turn carbonyl and Het is oxygen or NR¹¹,
   and are more preferably each independently hydrogen, C₁-C₁₂-alkyl, C₁-C₁₂-alkoxy, C₄-C₁₀-aryl.
R⁴ and R⁵ are preferably each independently selected from the group of fluorine, bromine, nitro, C₁-C₄-alkyl, C₁-C₄-alkoxy or radicals of the formulae (VIb) and (VIg),
   and are more preferably each identically selected from the group of fluorine and radicals of the formulae (VIb) and (VIg),
n and m are preferably each independently 0 or 1, n and m are more preferably each identically 0 or 1 and most preferably each 0.
R⁶ is preferably OR¹¹ or N(R¹¹)₂, where N(R¹¹)₂ together may also be a 5- or 6-membered cyclic amino radical, more preferably OR¹¹.

Preferably,
R⁷ and R⁸ are each independently hydrogen, C₁-C₄-alkyl, or C₂-C₈-alkenyl and
R⁹ and R¹⁰ are each independently hydrogen, C₁-C₄-alkyl, C₄-C₁₄-aryl or C₂-C₈-alkenyl, or alternatively
R⁸ and R¹⁰ in each case together are C₃-C₈-alkenediyl.

More preferably,
R⁷ and R⁹ are each independently hydrogen or C₁-C₄-alkyl and
R⁸ and R¹⁰ are in each case together C₃-C₈-alkenediyl.

Most preferably,
R⁷ and R⁹ are each identically hydrogen and
R⁸ and R¹⁰ are in each case together C₃-C8-alkenediyl.

Of compounds of the formula (I), very particular preference is given to those which bear radicals on the nitrogen atom which are selected from the group of

10-cyano-5H-dibenzo[a,d]cyclohepten-5-yl (^{CN}trop), 5H-dibenzo[a,d]cyclohepten-5-yl (trop), 10-methyl-5H-dibenzo[a,d]cyclohepten-5-yl (^{Me}trop), 10-methoxy-5H-dibenzo[a,d]cyclohepten-5-yl (^{MeO}trop), 10-phenyl-5H-dibenzo[a,d]cyclohepten-5-yl (^{Ph}trop), 10,11-diphenyl-5H-dibenzo[a,d]cyclohepten-5-yl (^{ph2}trop) [(5S)-10-[(-)-menthyloxy]-5H-dibenzo[a,d]cyclohepten-5-yl], (S-^{menthyloxy}trop) and [(5R)-10-[(-)-menthyloxy]-5H-dibenzo[a,d]cyclohepten-5-yl] (R-^{menthyloxy}trop).

To prepare compounds of the formula (I), compounds of the formula (IV)
H₂NR¹ (IV)
wherein R¹ is as defined above
are preferably reacted, optionally in the presence of organic solvent, with compounds of the formula (V) 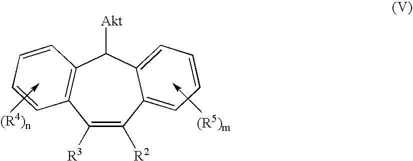
wherein R², R³, R⁴, R⁵ , n and m are each as defined above and
wherein Akt is Chlorine, Bromine, Fodine, Trifluoracetyl or a sulfonyloxy radical
the ammonium salts of the formula (VI) 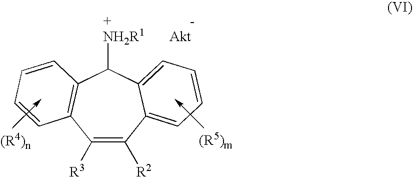
which are formed are preferably converted to compounds of the formula (I) in the presence of base.

The compounds of the formula (IV) and (V) are either known from the literature or can be synthesized analogously to the literature.

The reaction may optionally be, and is preferably, carried out in the presence of organic solvent. Suitable organic solvents are, for example:
aliphatic or aromatic, optionally halogenated hydrocarbons, for example various benzines, benzene, toluene, xylene, chlorobenzene, dichlorobenzene, various petroleum ethers, hexane, cyclohexane, dichloromethane, chloroform, carbon tetrachloride; ethers such as diethyl ether, methyl tert-butyl ether, diisopropyl ether, dioxane, tetrahydrofuran or ethylene glycol dimethyl ether or ethylene glycol diethyl ether, or mixtures of such organic solvents.

Suitable bases are, for example: alkaline earth metal or alkali metal hydrides, hydroxides, amides, alkyl-substituted disilylamides, dialkylamides, alkoxides or carbonates, for example sodium hydride, sodium amide, lithium diethylamide, sodium methoxide, sodium bistrimethylsilylamide, sodium ethoxide, potassium tert-butoxide, sodium hydroxide, potassium hydroxide, sodium carbonate and potassium carbonate, tertiary amines such as trimethylamine, triethylamine, tributylamine, trioctylaamine, diisopropylethylamine, tetramethylguanidine, N,N-diimethylaniline, diazabicyclooctane (DABCO), diazabicyclononene (DBN) or diazabicycloundecene (DBU), piperidine and N-methylpiperidine.

Both the preparation process for the compounds of the formula (I) and the compounds of the formula (VI) as indispensable intermediates are embraced fully by the invention.

The invention also includes transition metal complexes of compounds of the formula (I) and also catalysts which comprise the inventive transition metal complexes of compounds of the formula (I).

Preferred transition metal complexes are transition metal complexes of ruthenium, osmium, cobalt, rhodium, iridium, nickel, palladium, platinum and copper, preferably those of ruthenium, rhodium, iridium, nickel, palladium and platinum, more preferably those of rhodium and iridium.

The catalysts used may, for example, be isolated transition metal complexes which have been obtained, for example, from the compounds of the formula (I) and a metal compound, or transition metal complexes which are obtained from the compounds of the formula (I) and a metal compound in the reaction medium of the catalysis.

Suitable metal compounds are, for example and with preference, those of the formula (VUa)
M¹(Y¹)ₚ (VIIa)
wherein
M¹ is ruthenium, rhodium, iridium, nickel, palladium, platinum or copper and
Y¹ is chloride, bromide, acetate, nitrate, methanesulfonate, trifluoromethanesulfonate or acetylacetonate and
p is 3 in the case of ruthenium, rhodium and iridium, is 2 in the case of nickel, palladium and platinum, and is 1 in the case of copper,
   or metal compounds of the general formula (VIIb)
   M¹(Y²)ₚB¹₂ (VUb)
   wherein
Y² is an anion, for example chloride, bromide, acetate, methanesulfonate, trifluoro-methanesulfonate, tetrafluoroborate, hexafluorophosphate, perchlorate, hexa-fluoroantimonate, tetra(bis-3,5-trifluoromethylphenyl)borate or tetraphenylborate and
p is 1 in the case of rhodium and iridium, is 2 in the case of nickel, palladium, platinum and ruthenium, and is 1 in the case of copper,
B¹ is in each case a C₂-C₁₂-alkene, for example ethylene or cyclooctene, or a nitrile, for example acetonitrile, benzonitrile or benzyl nitrile, or
B¹₂ together is a (C₄-C₁₂)-diene, for example norbomadiene or 1,5-cyclooctadiene
   or metal compounds of the formula (VIIc)
   [M²B²Y¹₂]₂ (VIIc)
   wherein
M² is ruthenium and
B² is aryl radicals, for example cymene, mesityl, phenyl or cyclooctadiene, norbornadiene or methylallyl
   or metal compounds of the formula (VIId)
   Meₚ[M³(Y³)₄] (VIId)
   where
M³ is palladium, nickel, iridium or rhodium and
Y³ is chloride or bromide and
Me is lithium, sodium, potassium, ammonium or organic ammonium and
p is 3 in the case of rhodium and iridium, or is 2 in the case of nickel, palladium and platinum
   or metal compounds of the formula (VIIe)
   [M⁴(B³)₂]An (VIIe),
   where
M⁴ is iridium or rhodium and
B³ is a (C₄-C₁₂)-diene, for example norbomadiene or 1,5-cyclooctadiene
An is a noncoordinating or weakly coordinating anion, for example methanesulfonate, trifluoromethanesulfonate, tetrafluoroborate, hexafluorophosphate, perchlorate, hexafluoroantimonate, tetra(bis-3,5-trifluoromethylphenyl)borate or tetraphenylborate.

Suitable metal compounds are additionally, for example, Ni(1 ,5-cyclooctadiene)₂, Pd₂(dibenzylideneacetone)₃, Pd[PPh₃]₄ cyclopentadienyl₂Ru, Rh(acac)(CO)₂, [RhCl(CO)₂]; Ir(pyridine)₂(1,5-cyclooctadiene), Ir(acac)(CO)₂, [IrCl(CO)₂], Cu(phenyl)Br, Cu(phenyl)Cl, Cu(phenyl)I, Cu(PPh₃)₂Br, [Cu(CH₃CN)₄]BF₄ and [Cu(CH₃CN)₄]PF₆ or polynuclear bridged complexes, for example [Rh(1,5-cyclooctadiene)Cl]₂ and [Rh(1,5-cyclooctadiene)Br]₂, [Rh(ethene)₂Cl]₂, [Rh(cyclooctene)₂Cl]₂.

The metal compounds used are preferably:

[Rh(COD)Cl]₂, [Rh(COD)₂Br], [Rh(COD)₂]ClO₄, [Rh(COD)₂]BF₄, [Rh(COD)₂]PF₆, [Rh(COD)₂]OTf, [Rh(COD)₂]BAr₄ (Ar=3,5-bistrifluoromethylphenyl) [Rh(COD)₂]SbF₆ RuCl₂(COD), [(cymene)RuCl₂]₂, [(benzene)RuCl₂]₂, [(mesitylene)RuCl₂]₂, [(cymene)RuBr₂]₂, [(cymene)RuI₂]₂, [(cymene)Ru(BF₄)₂]₂, [(cymene)Ru(PF₆)₂]₂, [(cymene)Ru(BAr₄)₂]₂, (Ar=3,5-bistrifluoromethylphenyl), [(cymene)Ru(SbF₆)₂]₂, [Ir(COD)₂Cl]₂, [lr(COD)₂]PF₆, [Ir(COD)₂]ClO₄, [Ir(COD)₂]SbF₆ [Ir(COD)₂]BF₄, [Ir(COD)₂]OTf, [Ir(COD)₂]BAr₄ (Ar=3,5-bistrifluoromethylphenyl), RuCl₃, NiCl₂, RhCl₃, PdCl₂, PdBr₂, Pd(OAc)₂, Pd₂(dibenzylideneacetone)₃, Pd(acetylacetonate)₂, Rh(acetylacetonate)(CO)₂, [RhCl(CO)₂]; Ir(pyridine)₂(COD), Ir(acac)(CO)₂, [IrCl(CO)₂] [Rh(nbd)Cl]₂, [Rh(nbd)₂Br], [Rh(nbd)₂]ClO₄, [Rh(nbd)₂]BF₄, [Rh(nbd)₂]PF₆, [Rh(nbd)₂]OTf, [Rh(nbd)₂]BAr₄ (Ar=3,5-bistrifluoromethylphenyl) [Rh(nbd)₂]SbF₆ RuCl₂(nbd), [Ir(nbd)₂]PF₆, [Ir(nbd)₂]ClO₄, [Ir(nbd)₂]SbF₆ [Ir(nbd)₂]BF₄, [Ir(nbd)₂]OTf, [Ir(nbd)₂]BAr₄ (Ar=3,5-bistrifluoromethylphenyl), Ir(pyridine)₂(nbd), [Ru(DMSO)₄Cl₂], [Ru(CH₃CN)₄Cl₂], [Ru(PhCN)₄Cl₂], [Ru(COD)Cl₂]ₙ, [Ru(COD)(methallyl)₂], [Ru(acetylacetonate)₃].

Even greater preference is given to Rh(acetylacetonate)(CO)₂, [RhCl(CO)₂] and Ir(acetylacetonate)(CO)₂, [IrCl(CO)₂].

The amount of the metal compound used may, based on the metal content, be, for example, 25 to 200 mol % in relation to the compound of the formula (I) used; preference is given to 80 to 140 mol %, very particular preference to 90 to 120 mol % and even greater preference to 95 to 105 mol %.

Very particularly preferred transition metal complexes of compounds of the formula (I) are those which obey the formula (VIII)
[M⁵(I)(L)]An (VIII)
wherein, in each case,
M⁵ is rhodium or iridium
(I) is a compound of the formula (I)
(L) is an uncharged mono- or bidentate ligand and
An without regard for a possible coordination to the metal, is an anion, for example chloride, bromide, iodide, methanesulfonate, trifluoromethanesulfonate, tetrafluoroborate, hexafluorophosphate, perchlorate, hexafluoroantimonate, tetra(bis-3,5-trifluoro-methylphenyl)borate or tetraphenylborate.

Preferred uncharged monodentate ligands are, for example, olefins such as cyclohexene, carbonyl, nitriles, for example acetonitrile or benzonitrile, phosphines, for example tri(C₄-C₁₄-aryl)phosphines, tri(C₁-C₈-alkyl)phosphines, bis(C₄-C₁₄-aryl)(C₁-C₈-aIkyl)phosphines and (C₄-C₁₄-aryl)di(C₁-C₈-alkyl)phosphines and phosphites, for example tri(C₄-C₁₄-aryl)phosphites, tri(C₁-C₈-alkyl)phosphites, bis(C₄-C₁₄-aryl)(C₁-C8-alkyl)phosphites and (C₄-C₁₄-aryl)di(C₁-C₈-alkyl)phosphites.

Preferred uncharged bidentate ligands are, for example, diolefms such as 1,5-cyclooctadiene, norbornadiene, dinitrogen compounds, for example 2,2′-bipyridine, and diphosphorus compounds, for example those of the formula (IX)
(R¹³-E)₂P-E-Z-E-P(E-R¹³)₂ (IX)
wherein
E is in each case independently, and independently of R¹³ and Z, absent or is oxygen and
   the R¹³ radicals are each independently C₁-C8-alkyl or are unsubstituted, mono-, di- or tri-R¹⁴-substituted phenyl, naphthyl or heteroaryl having 5 to 12 skeleton carbon atoms, where
   R¹⁴ is in each case independently selected from the group of C₁-C₈-alkyl, C₁-C₈-alkoxy, fluorine or cyano and
Z is an unsubstituted or substituted radical from the group of C₁-C₄-alkylene, 1,2-phenylene, 1,3-phenylene, 1,2-cyclohexyl, 1,1′-ferrocenyl, 1,2-ferrocenyl, 2,2′-(1,1′-binaphthyl) and 1,1′-biphenylyl.

Catalysts which comprise the inventive transition metal complexes are suitable especially for use in homogeneous catalysis.

It is common to the transition metal complexes specified that the proton which is bonded to the nitrogen atom of the central 7-membered ring of the compound of the formula (I) is very acidic and can be removed readily by base.

Such transition metal complexes are likewise suitable for use as a catalyst and, according to the applicant's findings, also constitute intermediates of the catalytic cycle.

The invention therefore further embraces transition metal complexes which comprise the compounds of the formula (I) in such a way that the nitrogen atom of the central 7-membered ring of compounds of the formula (I) is coordinated to the metal atom as an amide.

For such complexes, the above-specified areas of preference for the compounds of the formula (I) and the transition metal complexes apply analogously.

Particularly preferred transition metal complexes of this type are accordingly those of the formula (X)
[M⁶(I-)(L)] (X)
wherein, in each case,
M⁶ is rhodium or iridium
(I-) is a compound of the formula (I) wherein the nitrogen atom of the central 7-membered ring coordinates to the metal atom as an amide and
(L) is an uncharged mono- or bidentate ligand.

Preference is given to using the inventive catalysts and transition metal complexes for hydrogenations, more preferably for asymmetric hydrogenations, when the compounds are chiral under the prerequisites stated at the outset.

Preferred hydrogenations are, for example, hydrogenations of prochiral C═C bonds, for example prochiral enamines, olefins, enol ethers, C═O bonds, for example prochiral ketones, and C═N bonds, for example prochiral imines. Particularly preferred asymmetric hydrogenations are hydrogenations of C═O bonds, for example prochiral ketones.

In a preferred embodiment, the hydrogenation is carried out in the presence of a hydrogen donor molecule and optionally of a base.

Hydrogen donor molecules are, for example, molecular hydrogen, formic acid, ethanol or isopropanol; bases are, for example, alkoxides or tertiary amines. Particularly preferred mixtures of hydrogen donor molecule and base are mixtures of formic acid and triethylamine, in particular the azeotropic mixture thereof, and mixtures of potassium isopropoxide and isopropanol.

The amount of the metal compound used or of the transition metal complex used may, based on the particular metal content, be, for example, 0.001 to 20 mol %, based on the substrate used, preferably 0.001 to 2 mol %, most preferably 0.001 to 1 mol %.

In a preferred embodiment, asymmetric hydrogenations may be carried out, for example, in such a way that the catalyst is generated in situ from a metal compound and a compound of the formula (I), optionally in a suitable organic solvent, the substrate is added and the reaction mixture, at reaction temperature, is either placed under hydrogen pressure or admixed with a mixture of another hydrogen donor molecule and a base.

The inventive catalysts are suitable in particular in a process for preparing active ingredients of medicaments and agrochemicals, or intermediates of these two classes.

The advantage of the present invention lies in the possibility of preparing a whole class of high-performance catalysts using readily obtainable compounds which can be handled without risk.

### EXAMPLES

### Example 1

Synthesis of bis(5H-dibenzo[a,d]cyclohepten-5-yl)amine [trop₂NH]

3 g of 5H-dibenzo[a,d]cyclohepten-5-yl chloride (13.2 mmol) were dissolved in 60 ml of toluene and admixed with 1 ml (1.41 g; 8.7 mrnmol; 30% excess) of 1,1,1,3,3,3-hexamethyldisilazane. The mixture was heated under reflux for 4 h. Subsequently, all volatile constituents were removed under reduced pressure. Subsequently, 30 ml of hexane were added and the resulting suspension was heated. In the course of this, a white powder precipitated out with a yield of 95% of theory.

### Example 2

Synthesis of [Rh{(trop)₂NH}Cl]₂

0.47 g (1.2 mmol) of bis(5H-dibenzo[a,d]cyclohepten-5-yl)amine according to Example 1 and 0.28 g (0.56 mmol) of di-μ-chlorobis[(η-1,5-cyclooctadiene)rhodium(I)] were dissolved in 7 ml of dichloromethane and left to stand for two days. In the course of this, dark red crystals grew. The supernatant solvent was decanted. The crystals were washed with dichloromethane and dried under reduced pressure.

### Example 3

Synthesis of [Rh{(trop)₂NH}(CO)Cl] (3a) and [Rh{(trop)₂NH}(CO)]OTf (3b)

0.63 g (0.59 mmol) of di-μ-chlorobis[(bis(5H-dibenzo[a,d]cyclohepten-5-yl)amine)rhodium(I)] from Example 2 was suspended in THF. The air was removed from the flask. Carbon monoxide was then passed through the suspension, whereupon the cloudy orange solution became clear and yellow. This afforded 3a. After 0.31 g (1.2 mmol) of silver triflate had been added, the solution became red. After 24 hours, the silver chloride precipitate was filtered off, the solution was partly concentrated and the complex 3b was precipitated with hexane as an orange powder. Yield: 0.58 g (86%).

### Example 4

Synthesis of [Rh{(trop)₂NH}(PPh₃)Cl]

0.16 g (0.15 mmol) of di-μ-chlorobis[(bis(5H-dibenzo[a,d]cyclohepten-5-yl)amine)rhodium(I)] and 0.09 g (0.3 mmol) of triphenylphosphine were suspended in 20 ml of THF. The suspension became a clear solution at 60° C. Approx. 30 ml of toluene and 100 ml of hexane were added, whereupon (bis(5H-dibenzo[a,d]cyclohepten-5-yl)amine)chloro(triphenylphosphine)rhodium(I) precipitated out.

This was filtered off. Yield: 0.17 g (70%).
¹H NMR (250 MHz, CDCl₃) δ=0.4 (s b, 1H, amine) 3.8 (s, 2 H, benzyl) 5.3 (t, J=7.5 Hz, 2 H, olefin) 5.4 (t, J=8.5 Hz, 2 H, olefm) 5.6 (ddd, J=9.4, 5.7, 1.5 Hz, 2 H, phenyl) 6.5 (d, J=6.8 Hz, 2 H, arom) 6.6-7.3 (m, 17 H, arom) 7.4 (d, J=5.3 Hz, 6 H, arom) 8.1 (m, 4 H, phenyl)
P NMR (101 MW) 6=7.7 (d, J=110.9 Hz, 1 P)

### Example 5

Synthesis of [Rh{(trop)₂NH}(PPh₃)]OTf

0.17 g (0.21 mmol) of (bis(5H-dibenzo[a,d]cyclohepten-5-yl)amine)chloro(triphenyl-phosphine)rhodium(I) from Example 4 and 0.06 g (0.3 mmol) of silver triflate were stirred at 60° C. for hajlf an hour. The suspension was filtered through a filter paper. The product was precipitated from the mother liquor using hexane. Yield: 1.47 g (70%)
¹H NMR (250 MHz, CDCl₃) δ =5.0 (d, J=8.7 Hz, 4 H, 2 olefin, 2 benzyl) 5.5 (td, J=9.4, 3.0 Hz, 2 H, olefin) 5.7 (d, J=5.3 Hz, 1 H, amine) 6.8 (m, 8 H, arom) 7.3 (m, 8 H, arom) 7.6 (s, 9 H, m u. p phenyl ) 7.9 (m, 6 H, o phenyl)
³¹p NMR (101 MHz, CDCl₃) δ =38.8 (d, J=138.0 Hz, 1 P)

### Example 6

Synthesis of [Rh{(trop)₂NH}(P(OCH₃)₃]OTf

500 mg of the complex from Example 3b (0.74 mmol) were dissolved in 60 ml of THF and admixed with 0.5 ml of trimethyl phosphite. When this was done, the colour of the solution changed from yellow to pale yellow and yellow needles of the desired product precipitated out after 2 hours. Yield: 99% of theory.
¹H NMR (400.1 MHz, CD₂Cl₂) δ =3.34 (d, ³J_{PH}=10.5 Hz, 9 H, H¹²), 3.99 (d, ³J_{HH}=10.7 Hz, 9 H, H¹³), 4.17 (d, ³J_{PH}=5.7 Hz, 1 H, NH), 4.83 (d, ⁴J_{PH}=13.0 Hz, 2 H, H⁵), 5.15 (m, 2 H, H¹¹), 5.17 (m, 2 H, H¹⁰), 6.90 (d, ³J_{HH}=7.8 Hz, 2 H, H⁴), 6.91 (dd, ³J.=7.7 Hz, ³J_{HH}=7.7 Hz, 2 H, H²), 6.97 (m, 2 H, H³), 6.99 (m, 2 H, H¹), 7.20 (ddd, ³J_{HH}=7.4 Hz, ³J_{HH}=7.4 Hz, ⁴J_{HH}=1.2 Hz, 2 H, H⁷), 7.28 (ddd, ³J_{HH}=7.5 Hz, ³J_{HH}=7.5 Hz, ⁴J_{HH}=1.4 Hz, 2 H, H⁸), 7.30 (dd, ³JH=7.4 Hz, ⁴J_{HH}=1.4 Hz, 2 H, H⁶), 7.49 (dd, ³J_{HH}=7.53 Hz, ⁴J_{HH}=1.1 Hz, 2 H, H⁹)
³¹p NMR (162.0 MHz, CD₂Cl₂) 6=112.1 (dd, ¹J_{Rhp}=190 Hz, ²J_{PP}=68 Hz, Paxiai), 124.5 (dd, ¹J_{Rhp}=200 Hz, ²J_{PP}=68 Hz, Pequat)

### Example 7

Synthesis of [Rh{(trop)₂N-}(PPh₃]

200 mg of the complex from Example 5 were suspended in 50 ml of THF and admixed with 30 mg (0.267 mmol) of KOtBu. When this was done, the colour of the solution changed to green and it homogenized within 30 minutes. After filtration of the precipitated KOTf and drying under reduced pressure, the desired product was obtained in a virtually quantitative yield.
¹H NMR (400.1 MWz, THF-d8, 200 K) δ =4.69 (ddd, ³J_{HH}=9.0 Hz, ³J_{PH}=6.2 Hz,²J_{RhH}=1.2 Hz, 2 H, H¹⁰), 4.92 (d, ⁴J_{PH}=13.5 Hz, 2 H, H⁵), 5.62 (ddd,³J_{HH}=9.0 Hz, ²J_{RhH}=3.3 Hz, ³J_{PH}=2.9 Hz, 2 H, H¹¹), 6.57 (dd, ³J_{HH}=7.3 Hz, ³J_{HH}=7.3 Hz, 2 H, H²), 6.67 (dd, ³J_{HH}=7.2 Hz, ³J_{HH}=7.2 Hz, 2 H, H³), 6.79 (d, ³JH=7.6 Hz, 2 H, H⁴), 6.90 (d, ³J_{HH}=7.3 Hz, 2 H, H⁴), 6.95 (d, ³J_{HH}=7.0 Hz, 2 H, H⁹), 7.03 (m, 4 H, H⁷/H⁸), 7.22 (d, ³J_{HH}=6.7 Hz, 2 H, H⁶), 7.56 (m, 9 H, m-PPh₃/p-PPh₃), 7.63 (m, 6 H, o-PPh₃)

### Example 8

Hydrogenations with [Rh{(trop)₂N-}(PPh₃] from Example 7

A solution of 5 mg (0.0066 mmol) of the complex from Example 7 in 5 ml of THF was admixed with 129 mg of cyclohexanone (1.31 mmol, 200 eq.) and stirred under a hydrogen atmosphere (4 bar) over 18 h. Gas chromatography of the reaction solution showed complete conversion to cyclohexanol.

### Examples 9 to 23

Hydrogenations with the Complexes from Examples 3b, 5 and 6

| | Catalyst | | | | | | | |
|---|---|---|---|---|---|---|---|---|
| | from | | C/S^{a} | | | TOFᵢ^{c} | TOFₜ^{d} | tₘₐₓ^{e} [h] |
| | Example | Substrate | ppm | Alcohol | TON^{b} | [l/h] | [l/h] | (Cₘₐₓ [%]) |
| 9 | 3b | Cyclohexanone | 1000 | iPrOH | 950 | 1700 | 640 | 1.5 (97) |
| 10 | 3b | Cyclohexanone | 250 | iPrOH | 3900 | 990 | 420 | 9.2 (96) |
| 11 | 3b | Cyclohexanone | 730 | iPrOH | 1400 | 1300 | 370 | 3.7 (98) |
| 12 | 3b | Cyclohexanone | 250 | EtOH | 3800 | 3300 | 1900 | 2.0 (100) |
| 13 | 3b | Acetophenone | 980 | iPrOH | 760 | 530 | 28 | 27.0 (74) |
| 14 | 3b | Benzo- | 990 | iPrOH | 360 | 850 | 40 | 9.0 (35) |
| | | phenone | | | | | | |
| 15 | 3b | Benzo- | 4000 | iPrOH | 290 | 1200 | 35 | 9.2 (61) |
| | | phenone | | | | | | |
| 16 | 6 | Cyclohexanone | 980 | iPrOH | 1000 | — | 4100 | <0.25 (100) |
| 17 | 6 | Cyclohexanone | 63 | iPrOH | 15300 | 23900 | 46000 | 0.3 (97) |
| 18 | 6 | Cyclohexanone | 4.4 | iPrOH | 18100 | 15400 | 120000 | 3.7 (79) |
| 19 | 6 | Benzo- | 1000 | iPrOH | 940 | 9500 | 1100 | 0.8 (97) |
| | | phenone | | | | | | |
| 20 | 5 | Cyclohexanone | 1000 | iPrOH | 1000 | — | 4000 | <0.25 (100) |
| 21 | 5 | Cyclohexanone | 49 | iPrOH | 20300 | 126000 | 120000 | 0.18 (100) |
| 22 | 5 | Cyclohexanone | 49 | EtOH | 20250 | 22000 | 81000 | <0.25 (100) |
| 23 | 5 | Acetophenone | 49 | EtOH | 20100 | 79000 | 60000 | 0.3 (100) |

^{a}C/S = (amount of catalyst) × 10⁶/(amount of substrate),
^{b}TON = (amount of product)/(amount of catalyst) specified at the end of the catalysis, or the time at which the reaction was terminated,
^{c}TOF at the start of the catalysis,
^{d}TOF at the end of the catalysis,
^{e}time after which maximum conversion was attained. The maximum attained conversion is in brackets afterwards.

### Example 24

Synthesis of Methyl N-(5H-dibenzo[a,d]cyclohepten-5-yl)-L-2,5-cyclohexa-dienylalanate (trop-cyclohexadienylalanine)

0.42 g (4.2 nunol) of triethylamine was added dropwise to 0.915 g (4.2 mmol) of methyl cyclohexadienylalanate in 10 ml of dryCH₂Cl₂. After stirring for 30 minutes, 0.952 g (4.2 mmol) of 5H-dibenzo[a,d]cyclohepten-5-yl chloride was added via a powder funnel and a further 2.0 g of triethylamine were added in one portion. After stirring for a further 2 h, the organic phase was washed with 2×10 ml of water, dried over MgSO₄ and filtered. After the solvent had been removed under reduced pressure, 1.52 g (97%) of the product were obtained as a colourless oil.
¹H NMR (300 MHz, 25° C., CDCl₃): δ 7.85-7.30 (m, 8H), 7.08 (dd, 2H), 5.87-5.74 (m, 2H), 5.50 (s, 1H), 5.00 (s, 1H), 3.85, 3.74 (each s, together 3H), 3.11 (dd, 1H), 2.90-2.00 (m, 7H) ppm.

### Example 25

Synthesis of [Rh(trop-cyclohexadienylalanine)(CO)Cl]

97 mg (0.25 mmol) of [Rh(CO)₂Cl]₂ were added at room temperature to a solution of 220 mg (0.59 mmol) of the ligand from Example 24 in 10 ml of dry CH₂Cl₂. In the course of this, vigorous evolution of carbon monoxide was observed. The solution was concentrated to about 3 ml and blanketed with 10 ml of hexane. Overnight, 245 mg (92%) of the complex crystallized in the form of a pale yellow crystals.
¹H NMR (300 MHz, 25° C., CDCl₃): δ 7.60-7.05 (m, 8H), 5.77 (dm, ³J (H,H)=9.9 Hz, 1H:), 5.64 (dm, ³J (H,H)=9.9 Hz, 1H), 5.24 (dd, ³j (H,H)=9.3 Hz, ²J (Rh,H)=1.2 Hz, 1H), 5.15 (dd, ³J (H,H)=9.3 Hz, ²J (Rh,H)=2.4 Hz, 1H), 4.43 (br, 1H), 4.39 (s, 1H), 3.84 (br, 1H), 3.79 (s, 3H), 3.45 (m, 1H), 2.92-2.38 (m, 6H) ppm.

### Example 26

Synthesis of [Rh(trop-cyclohexadienylalanine)(CO)]OTf

64 mg (0.25 mmol) of silver triflate were added at room temperature to a solution of 119 mg (0.22 mol) of the complex from Example 25 in 10 ml of dry CH₂Cl₂. After stirring for 2 hours, the solution was filtered through Celite, concentrated to about 3 ml and blanketed with 10 ml of hexane. Overnight, the product crystallized in virtually quantitative yield.
¹H NMR (300 MHz, 25° C., CDCl₃): δ 7.68-7.22 (m, 8H), 5.91 (dm, ³J (H,H)=10.5 Hz, 1H), 5.75 (dm, ³J (H,H)=10.5 Hz, 1H), 5.68 (dd, ³J (H,H)=9.0 Hz, ²j (Rh,H)=3.0 Hz, 1H), 5.45 (dd, ³J (H,H)=9.0 Hz, ²J (Rh,H)=0.9 Hz, 1H), 4.64 (br, 1H), 4.47 (br, 1H), 4.43 (br, 1H), 3.87 (s, 3H), 3.38-2.50 (m, 7H) ppm.

### Examples 27 and 28

Hydrogenations with [[Rh{trop-cyclohexydienylalanine}(CO)]OTf from Example 26

| Amount of | | | | | Enantiomeric |
|---|---|---|---|---|---|
| catalyst | Temp. | Time | | | excess |
| [mol %] | [° C.] | [min] | Substrate | Conversion | [% ee] |
| 0.1 | 25 | 25 | Acetophenone | 40% | 58 |
| 0.1 | 25 | 100 | Acetophenone | 77% | 58 |

Reaction medium: 2.0 g of acetophenone, 5 mM solution of potassium isopropoxide in 16.6 ml of isopropanol

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Compounds of the formula (I) 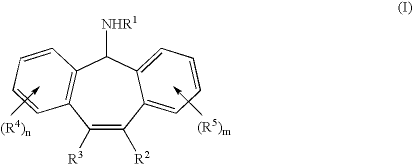
wherein
R¹ is either a radical of the formula (II) 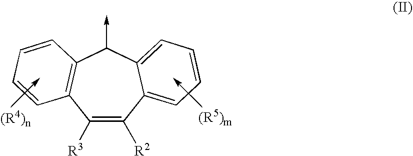
or is a radical of the formula (III) 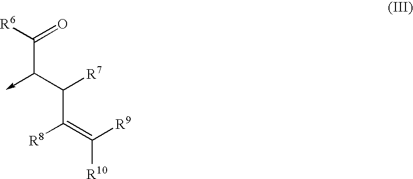
where, in the formulae (I), (II) and (III),
the arrows each indicate the bond of the overall radical to the nitrogen atom or, when they point into the middle of an aromatic system, a bond of the particular radicals to the aromatic skeleton in any position,
R² and R³ are each independently hydrogen, cyano, fluorine, chlorine, bromine, iodine, C₁-C₁₈-alkyl, C₄-C₂₄-aryl, C₅-C₂₅-arylalkyl, CO₂M where M may be an alkali metal ion or an optionally organic ammonium ion, CONH₂, SO₂N(R¹¹)₂ where R¹¹ is hydrogen, C₁-C₁₂-alkyl, C₄-C₁₄-aryl or C₅-C₁₅-arylalkyl, SO₃M or are radicals of the formula (IV),
T-Het-R¹² (IV)
wherein
T is absent or is carbonyl,
Het is oxygen or NR¹¹,
R¹² is C₁-C₁₈-alkyl, C₄-C₂₄-aryl or C₅-C₂₅-arylalkyl or N(R¹²)₂ as a whole is a 5- or 6-membered cyclic amino radical and
n and m are each independently 0, 1, 2 or 3 and
R⁴ and R⁵ are each independently selected from the group of fluorine, chlorine, bromine, iodine, nitro, free or protected formyl, C₁-C₁₂-alkyl, C₁-C₁₂-alkoxy, C₁-C₁₂-haloalkoxy, C₁-C₁₂-haloalkyl, C₄-C₁₄-aryl, C₅-C₁₅-arylatkyl or radicals of the formula (V)
L-Q-T-W (V)
wherein, each independently,
L is absent or is C₁-C₈-alkylene or C₂-C₈-alkenylene and
Q is absent or is oxygen, sulfur or NR¹¹,
T is a carbonyl group and
W is R¹¹, OR¹¹, NHR12 or N(R¹²)₂, where N(R¹²)₂ as a whole may also be a 5- or 6-membered cyclic amino radical,
or radicals of the formulae (VIa-g)
L-W (VIa) L-SO₂-W (Vlb)
L-NR¹²SO₂R¹² (VIc) L-SO₃Z (VId)
L-PO₃Z₂ (VIe) L-COZ (VIf)
L-CN (VIg)
wherein L, Q, W and R¹² are each as defined under formula (IV) and Z is hydrogen or M
and, in formula (El),
R⁶ is OR¹¹ or N(R¹¹)₂, where N(R¹¹)₂ together may also be a 5- or 6-membered cyclic amino radical,
R⁷ and R⁸ are each independently hydrogen, C₁-C₈-alkyl, C₄-C₁₀-aryl, C₅-C₁₁-arylalkyl or C₂-C₈-alkenyl and
R⁹ and R¹⁰ are each independently hydrogen, C₁-C₈-alkyl, C₄-C₁₄-aryl, C₅-C₁₁-arylalkyl or C₂-C₈-alkenyl or
R⁷ and R⁹ or R⁸ and R¹⁰, in each case together, are C₃-C₁₂-alkylene or C₃-C₁₂-alkenylene.

2. Process for preparing compounds of the formula (I) according to claim 1 comprising reacting compounds of the formula (IV)
H₂NR¹ (IV)
wherein R¹ is as defined in claim 1
with compounds of the formula (V) 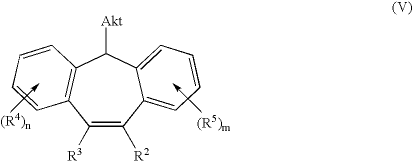
wherein R², R³, R⁴, R⁵, n and m are each as defined in claim 1 and
wherein Akt is Chlorine, Bromine, Fodine, Trifluoracetyl or a sulfonyloxy radical the ammonium salts of the formula (VI) 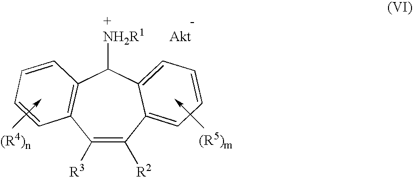
which are formed are converted to compounds of the formula (I) in the presence of base.

3. Compounds of the formula (VI) according to claim 2.

4. Transition metal complexes of compounds of the formula (I) according to claim 1.

5. Transition metal complexes according to claim 4, wherein they obey the formula (VIII)
[M⁵(I)(L)]An (VIII)
wherein
M⁵ is rhodium or iridium
(I) is a compound of the formula (I)
(L) is an uncharged mono- or bidentate ligand and
An, without regard for a possible coordination to the metal, is an anion.

6. Transition metal complexes comprising compounds of the formula (I) according to claim 1, in such a way that the nitrogen atom of the central 7-membered ring of the compounds of the formula (I) coordinates to the metal atom as an amide.

7. Transition metal complexes according to claim 6, wherein they obey the formula (X)
[M⁶(I-)(L)] (X)
wherein
M⁶ is rhodium or iridium
(I-) is a compound of the formula (I) wherein the nitrogen atom of the central 7-membered ring coordinates to the metal atom as an amide and
(L) is an uncharged mono- or bidentate ligand.

8. Catalysts comprising transition metal complexes according to claim 4, 5, 6 or 7.

9. Use of catalysts according to claim 8 for hydrogenations.
